(19) United States
(12) Patent Application Publication
(10) Pub. No.: US 2012/0231047 A1
(43) Pub. Date: Sep. 13, 2012

(54) THERAPEUTIC AGENT DELIVERY FOR THE TREATMENT OF ASTHMA VIA IMPLANTABLE AND INSERTABLE MEDICAL DEVICES

(75) Inventors: Robert A. Herrmann, Boston, MA (US); John J. Damarati, Marlborough, MA (US); Sepideh Hashemi, Weston, MA (US); Wendy Naimark, Boston, MA (US); Barry N. Gellman, North Easton, MA (US); Robert Rioux, Ashland, MA (US); Alexandra Rousseau, Cambridge, MA (US); Raymond Lareau, Westford, MA (US)

(73) Assignee: BOSTON SCIENTIFIC SCIMED, INC., Maple Grove, MN (US)

(21) Appl. No.: 13/480,227

(22) Filed: May 24, 2012

Related U.S. Application Data
(63) Continuation of application No. 11/726,608, filed on Mar. 22, 2007.
(60) Provisional application No. 60/839,752, filed on Aug. 24, 2006.

(51) Int. Cl. A61K 31/56; A61K 47/02; A61P 11/06; A61M 37/00; A61P 35/00; A61P 21/02; A61M 25/10; A61K 9/00; A61P 29/00

(52) U.S. Cl. 424/400; 514/169; 514/769; 604/509; 604/22; 604/103.02

(57) ABSTRACT

Methods for the treatment of asthma are provided, which comprise: (a) providing an implantable or insertable medical device that comprises an asthma treatment agent; and (b) inserting or implanting the medical device within the lungs (e.g., the trachea or the bronchial tree) of a patient, whereupon the therapeutic agent is delivered to the patient in an amount effective to reduce or eliminate the symptoms of asthma. Also disclosed herein are medical devices and kits for carrying out such methods.

### STATEMENT OF RELATED APPLICATION

This application claims the benefit of U.S. Provisional Patent Application Ser. No. 60/839,752, filed Aug. 24, 2006, entitled “Therapeutic Agent Delivery for the Treatment of Asthma via Implantable and Insertable Medical Devices”, which is incorporated by reference in its entirety herein.

### TECHNICAL FIELD

This invention generally relates to medical devices, and more particularly to implantable or insertable medical devices for the treatment of asthma.

### BACKGROUND INFORMATION

Respiration is a key component of human life. The lungs remove oxygen from air for transport via the blood to the entire body. Air entering the lungs must travel through bronchial tubes, which can open or close in response to many stimuli. For example, in conjunction with reactive airway disease such as asthma, bronchi constrict and plug with mucus. When this happens, the quantity of air entering and leaving the lungs is greatly impaired and oxygen starvation begins. Continued evolution of a constricted and mucus filled bronchial tree eventually becomes life threatening.

Treatment of asthma typically involves the use of inhaled drugs (e.g., drug delivery via an inhaler) and/or systemic delivery of drugs (e.g., drug delivery via injection or ingestion of the drug). Unfortunately, inhalers require frequent treatment, which can inconvenience the patient. Moreover, inhalers may not provide effective delivery to the site of inflammation when airways are blocked or constricted. Systemic treatment results in exposure to the administered drug at locations far removed from the site of interest (i.e., the lungs), which can lead to unwanted side effects.

### SUMMARY OF THE INVENTION

The above and other challenges of the prior art are addressed by embodiments of the present invention in which various methods for the treatment of asthma are provided. These methods comprise the following: (a) providing an implantable or insertable medical device that comprises an asthma treatment agent; and (b) inserting or implanting the medical device within the lungs (e.g., the trachea or the bronchial tree) of a patient, whereupon the therapeutic agent is delivered to the patient in an amount effective to reduce or eliminate the symptoms of asthma.

Examples of therapeutic agents include steroidal and non-steroidal anti-inflammatory agents, anti-proliferative agents, anti-macrophage agents, and muscle relaxants.

In some aspects of the invention, therapeutic agents are released from porous metallic or ceramic regions of the medical devices.

In some aspects of the invention, therapeutic agents are released from polymeric regions of the medical devices, which may be biostable or biodisintegrable. For example, in some embodiments, the polymeric regions are stand-alone polymeric matrices. In other embodiments the polymeric regions are polymeric matrices disposed as coatings on the medical devices. In still other embodiments, the polymeric regions are polymeric microparticles positioned at or near the surfaces of the medical devices. The polymeric microparticles may comprise, for example, (a) a polymeric matrix, which further comprises the therapeutic agent, or (b) a therapeutic agent containing core and a polymeric coating, which encapsulates the core. Examples of medical devices for use in these aspects of the present invention include stents, intraluminal paving systems, grafts and patches, among many others.

In other aspects of the invention, fluids that comprise the therapeutic agent are forced from the medical devices. In these aspects, the therapeutic agent may be disposed, for example, within solutions, suspensions, emulsions, or liposomal formulations, among others. Examples of medical devices for these aspects of the present invention include various catheters, for example, balloon catheters from which therapeutic agent is released upon expansion of the balloon and catheters for aerosol or spray delivery within the airways, as well as osmotic pumps, among various other devices.

In still other aspects of the invention, a fluid is applied, which is adapted to solidify subsequent to contact with the tissue of interest (e.g., in the formation of an intraluminal paving system).

Other embodiments of the invention are directed to medical devices, which comprise an asthma treatment agent selected from steroidal anti-inflammatory agents, non-steroidal anti-inflammatory agents, antiproliferative agents, muscle relaxants, anti-macrophage agents, nitric oxide donors, agents that stimulate production of nitric oxide in vivo, cGMP activators and anti-histamines, among others disclosed herein. Such medical devices are configured for implantation or insertion within the trachea or the bronchial tree of a patient, whereupon the asthma treatment agent is delivered to the patient in an amount effective to reduce or eliminate the symptoms of asthma.

Still other embodiments of the invention are directed to kits that comprise a composition comprising an asthma treatment agent selected from steroidal anti-inflammatory agents, non-steroidal anti-inflammatory agents, antiproliferative agents, muscle relaxants, anti-macrophage agents, nitric oxide donors, agents that stimulate production of nitric oxide in vivo, cGMP activators and anti-histamines, among others disclosed herein, and (b) a medical device configured for implantation or insertion within the trachea or the bronchial tree of a patient and for delivery of the composition to a patient in an amount effective to reduce or eliminate the symptoms of asthma.

One advantage of the present invention is that therapeutic agents may be locally delivered to the patient in an amount effective to reduce or eliminate the symptoms of asthma, thereby minimizing systemic effects and patient inconvenience.

These and other aspects, embodiments and advantages of the present invention will become immediately apparent to those of ordinary skill in the art upon review of the Detailed Description and Claims to follow.

### BRIEF DESCRIPTION OF THE DRAWINGS

FIG. 1 is a schematic illustration which shows three drug-releasing stents, disposed within the trachea, left primary bronchus and right primary bronchus of a subject, in accordance with an embodiment of the present invention.

### DETAILED DESCRIPTION OF THE INVENTION

Various aspects of the present invention are directed to implantable or insertable medical devices, which comprise an asthma treatment agent in an amount effective to reduce or eliminate the symptoms of asthma. The medical devices are adapted to be inserted or implanted at a desired site within the lungs (for example, the trachea and/or the bronchial tree, which contains the bronchi, including primary left and right bronchi, as well as branches thereof), whereupon the therapeutic agent is dispensed to the subject's lungs.

Subjects (also referred to herein as “patients”) for the procedures of the present invention include vertebrate subjects, typically mammalian subjects, and more typically human subjects.

In general, the therapeutic agents to be dispensed by the medical devices of the present invention include essentially any pharmaceutically acceptable therapeutic agent that is effective for the treatment of asthma. As used herein “pharmaceutically acceptable” means that an agent that is approved or capable of being approved by the United States Food and Drug Administration or Department of Agriculture for use in humans or animals. Examples of pharmaceutically acceptable therapeutic agents for use in conjunction with the present invention include anti-inflammatory agents, antiproliferative agents, anti-macrophage agents, muscle relaxants, nitric oxide donors, agents that stimulate production of nitric oxide, cGMP activators, anti-histamines, and combinations thereof. Also, supplementary agents such as narcotic and non-narcotic analgesics and local anesthetic agents are dispensed in some embodiments.

The amount of therapeutic agent that is dispensed by the medical devices of the present invention is a therapeutically effective amount. One skilled in the art can readily determine an amount of therapeutic agent that is therapeutically effective for the desired outcome (i.e., the treatment of asthma).

“Drugs,” “therapeutic agents,” “pharmaceutically active agents,” “pharmaceutically active materials,” and other related terms may be used interchangeably herein and include cells, genetic therapeutic agents, and non-genetic therapeutic agents. Therapeutic agents may be used singly or in combination.

Cells for use in conjunction with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophages, or cells from an animal, bacterial or fungal source (xenogeneic), which may be genetically engineered, if desired, to deliver proteins and other molecules of interest. Examples include cells that are genetically engineered to produce heme oxygenase, nitric oxide synthase (NOS) or superoxide dismutase, antiproliferative agents such as thymidine kinase, and anti-inflammatory agents such as interleukin-10.

Examples of genetic therapeutic agents for use in conjunction with the present invention include anti-sense DNA and RNA as well as DNA coding for: (a) anti-sense RNA, (b) tRNA or rRNA to replace defective or deficient endogenous molecules, (c) cell cycle inhibitors including CD inhibitors, (d) thymidine kinase (“TK”), and other agents useful for interfering with cell proliferation, and (e) anti-inflammatory agents such as interleukin-10.

Vectors for delivery of genetic therapeutic agents include (a) viral vectors such as adenoviruses, gutted adenoviruses, adeno-associated virus, retroviruses, alpha viruses (Semliki Forest, Sindbis, etc.), lentiviruses, herpes simplex viruses, replication competent viruses (e.g., ONYX-015) and hybrid vectors; and (b) non-viral vectors such as artificial chromosomes and mini-chromosomes, plasmid DNA vectors (e.g., pCOR), cationic polymers (e.g., polyethyleneimine (PEI), polyethyleneimine copolymers such as polyether-PEI and polyethylene oxide-PEI), neutral polymers PVP, SP1017 (SUPRATEK), lipids such as cationic lipids, liposomes, lipoplexes, microparticles, with and without targeting sequences such as the protein transduction domain (PTD).

Non-genetic therapeutic agents for use in conjunction with the present invention include both small molecule agents and biopolymers, such as polypeptide-containing therapeutic agents and polysaccharide-containing therapeutic agents, among others.

Anti-inflammatory agents include steroidal and non-steroidal anti-inflammatory agents. Examples of non-steroidal anti-inflammatory drugs include aminoarylcarboxylic acid derivatives such as enfenamic acid, etofenamate, flufenamic acid, isonixin, meclofenamic acid, mefanamic acid, niflumic acid, talniflumate, terofenamate and tolfenamic acid; arylacetic acid derivatives such as acemetacin, alclofenac, amfenac, bufexamac, cinmetacin, clopirac, diclofenac, etodolac, felbinac, fenclofenac, fenclorac, fenclozic acid, fentiazac, glucametacin, ibufenac, indomethacin, isofezolac, isoxepac, lonazolac, metiazinic acid, oxametacine, proglumetacin, sulindac, tiaramide, tolmetin and zomepirac; arylbutyric acid derivatives such as bumadizon, butibufen, fenbufen and xenbucin; arylcarboxylic acids such as clidanac, ketorolac and tinoridine; arylpropionic acid derivatives such as alminoprofen, benoxaprofen, bucloxic acid, carprofen, fenoprofen, flunoxaprofen, flurbiprofen, ibuprofen, ibuproxam, indoprofen, ketoprofen, loxoprofen, miroprofen, naproxen, oxaprozin, piketoprofen, pirprofen, pranoprofen, protizinic acid, suprofen and tiaprofenic acid; pyrazoles such as difenamizole and epirizole; pyrazolones such as apazone, benzpiperylon, feprazone, mofebutazone, morazone, oxyphenbutazone, phenybutazone, pipebuzone, propyphenazone, ramifenazone, suxibuzone and thiazolinobutazone; salicylic acid and its derivatives such as acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamine o-acetic acid, salicylsulfuric acid, salsalate and sulfasalazine; thiazinecarboxamides such as droxicam, isoxicam, piroxicam and tenoxicam; other agents such as ε-acetamidocaproic acid, s-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole and tenidap; as well as analogs, derivatives, and various salts of the foregoing.

Examples of steroidal anti-inflammatory agents (e.g., glucocorticoids, also known as corticosteroids) include 21-acetoxypregnenolone, alclometasone, algestone, amicinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumehtasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone, formocortal, halcinonide, halobetasol priopionate, halometasone, halopredone acetate, hydrocortamate, hydrocortisone, loteprednol etabonate, mazipredone, medrysone, meprednisone, methyolprednisolone, mometasone furoate, paramethasone, prednicarbate, prednisolone, prednisolone 25-diethylaminoacetate, prednisone sodium phosphate, prednisone, prednival, prednylidene, rimexolone, tixocortal, triamcinolone, triamcinolone acetonide, triamcinolone benetonide, and triamcinolone hexacetonide, as well as analogs, derivatives, and various salts of the foregoing.

Exemplary antiproliferative agents include anthracyclines, alkyl sulfonates, agents affecting microtubule dynamics, agents affecting various growth factors including IGF pathway agents such as somatostatin analogs, angiotensin converting enzyme inhibitors, antimetabolites (e.g., purine analogs), cytotoxic agents, cytostatic agents, cell proliferation affectors, caspase activators, proteasome inhibitors, angiogenesis inhibitors, ethylenimines, intercalating agents, metal coordination complexes, nitrogen mustards, nitrosoureas, nucleic acid damaging agents such as alkylating agents, purine analogs, pyrimidine analogs, inhibitors of pyrimidine biosynthesis and vinca alkaloids. Specific examples of antiproliferative compounds include adriamycin, alitretinoin (9-cis-retinoic acid), amifostine, angiopeptin, angiostatin, arabinosyl 5-azacytosine, arabinosyl cytosine, 5-aza-2′-deoxycytidine, 6-azacytidine, 6-azauridine, azaribine, bexarotene (4-[1-(5,6,7,8-tetrahydro-3,5,5,8,8-pentamethyl-2-naphthalenyl)ethenyl]benzoic acid), bleomycin, BCNU, CCNU, captopril, capecitabine (5′-deoxy-5-fluoro-cytidine), chlorambucil, colchicine, cilazapril, cisplatin, cladribine (a chlorinated purine nucleoside analog), cytarabine, cyclocytidine, cyclophosphamide, daunorubicin, 3-deazauridine, 2′-deoxy-5-fluorouridine, 5′-deoxy-5-fluorouridine, docetaxel, doxorubicin, endostatin, epirubicin, epothilone, estramustine, etoposide, exemestane, flutamide, fludarabine, fludarabin phosphate, fluorocytosine, 5-fluorouracil, 5-fluorouridine, 5-fluoro-2′-deoxyuridine, gemcitabine, hydroxyurea, idarubicin, irinotecan, LHRH analogs, lisinopril, melphalan, methotrexate, 6-mercaptopurine, mitoxantrone, ocreotide, paclitaxel, pentostatin, N-phosphonoacetyl-L-aspartic acid, prednimustine, pyrazofurin, squalamine, streptozocin, tamoxifen, temozolomide, teniposide, 6-thioguanine, tomudex, thiotepa, topotecan, 5-trifluoromethyl-2′-deoxyuridine, valrubicin, vincristine, vinblastine and vinarelbine, as well as analogs, derivatives, and various salts of the foregoing.

Exemplary anti-macrophage agents include bisphosphonates and chlodronate compounds, e.g., dichloromethylene diphosphonate (CL2MDP).

Examples of muscle relaxants include brochodilators, antispasmodics and antichoinergics. Examples of brochodilators include (a) ephedrine derivatives such as albuterol, bambuterol, bitolterol, carbuterol, clenbuterol, clorprenaline, dioxethedrine, ephedrine, epiniphrine, eprozinol, etafedrine, ethylnorepinephrine, fenoterol, hexoprenaline, isoetharine, isoproterenol, mabuterol, metaproterenol, n-methylephedrine, pirbuterol, procaterol, protokylol, reproterol, rimiterol, salmeterol, soterenol, terbutaline and tulobuterol; (b) quaternary ammonium compounds such as bevonium methyl sulfate, clutropium bromide, ipratropium bromide and oxitropium bromide; (c) xanthine derivatives such as acefylline, acefylline piperazine, ambuphylline, aminophylline, bamifylline, choline theophyllinate, doxofylline, dyphylline, enprofylline, etamiphyllin, etofylline, guaithylline, proxyphylline, theobromine, 1-theobromineacetic acid and theophylline; and (d) other bronchodilators such as fenspiride, medibazine, montekulast, methoxyphenanime, tretoquinol, zafirkulast, and cathcholamine analogs such as formoterol; as well as analogs, derivatives, and various salts of the foregoing.

Examples of antispasmodic agents include alibendol, ambucetamide, aminopromazine, apoatropine, bevonium methyl sulfate, bietamiverine, butaverine, butropium bromide, n-butylscopolammonium bromide, caroverine, cimetropium bromide, cinnamedrine, clebopride, coniine hydrobromide, coniine hydrochloride, cyclonium iodide, difemerine, diisopromine, dioxaphetyl butyrate, diponium bromide, drofenine, emepronium bromide, ethaverine, feclemine, fenalamide, fenoverine, fenpiprane, fenpiverinium bromide, fentonium bromide, flavoxate, flopropione, gluconic acid, guaiactamine, hydramitrazine, hymecromone, leiopyrrole, mebeverine, moxaverine, nafiverine, octamylamine, octaverine, oxybutynin chloride, pentapiperide, phenamacide hydrochloride, phloroglucinol, pinaverium bromide, piperilate, pipoxolan hydrochloride, pramiverin, prifinium bromide, properidine, propivane, propyromazine, prozapine, racefemine, rociverine, spasmolytol, stilonium iodide, sultroponium, tiemonium iodide, tiquizium bromide, tiropramide, trepibutone, tricromyl, trifolium, trimebutine, n,n-1trimethyl-3,3-diphenyl-propylamine, tropenzile, trospium chloride, and xenytropium bromide, as well as analogs, derivatives, and various salts of the foregoing.

Examples of anticholinergics include adiphenine, alverine, ambutonomium, aminopentamide, amixetrine, amprotropine phosphate, anisotropine methylbromide, apoatropine, atropine, atropine n-oxide, benactyzine, benapryzine, benzetimide, benzilonium, benztropine mesylate, bevonium methyl sulfate, biperiden, butropium, n-butylscopolammonium bromide, buzepide, camylofine, caramiphen, chlorbenzoxamine, chlorphenoxamine, cimetropium, clidinium, cyclodrine, cyclonium, cycrimine, deptropine, dexetimide, dibutoline sulfate, dicyclomine, diethazine, difemerine, dihexyverine, diphemanil methylsulfate, n-(1,2-diphenylethyl)nicotinamide, dipiproverine, diponium, emepronium, endobenzyline, ethopropazine, ethybenztropine, ethylbenzhydramine, etomidoline, eucatropine, fenpiverinium, fentonium, butropium, glycopyrrolate, heteronium, hexocyclium methyl sulfate, homatropine, hyoscyamine, ipratropium, isopropamide, levomepate, mecloxamine, mepenzolate, metcaraphen, methantheline, methixene, methscopolamine, octamylamine, oxybutynin, oxyphencyclimine, oxyphenonium, pentapiperide, penthienate, phencarbamide, phenglutarimide, pipenzolate, piperidolate, piperilate, poldine methysulfate, pridinol, prifinium, procyclidine, propantheline, propenzolate, propiverine, propyromazine, scopolamine, scopolamine n-oxide, stramonium, sultroponium, thiphenamil, tiemonium, timepidium, tiquizium, tridihexethyl iodide, trihexyphenidyl hydrochloride, trimebutine, tropacine, tropenzile, tropicamide, trospium, valethamate, vamicamide and xenytropium, as well as analogs, derivatives, and various salts of the foregoing.

Other muscle relaxants include alcuronium, atracurium, baclofen, benzodiazepines (e.g., clozapine or diazepam), botulinum toxin (BOTOX), 4-amino-3-(4-chloropheyl)-butanoic acid, carbolonium, carisoprodol, chlorphenesin, chlorzoxazone, cyclobenzaprine, cyclandelate, dantrolene, decamethonium bromide, diazepam hydralazine, fazadinium, gallamine, guaifenesin, hexafluorenium, isoxsuprine, meladrazine, mephensin, metaxalone, methocarbamol nylidrin, metocurine iodide, orphenadrine, pancuronium, papaverine, pridinol, styramate, suxamethonium, suxethonium, thiocolchicoside, tizanidine, suxamethonium, tolperisone and tubocurarine, as well as analogs, derivatives, and various salts of the foregoing.

Other anti-asthmatic agents, not necessarily exclusive of those above, include amlexanox, aminophylline, azelastine, beclometaason dipropionate, cromolyn, dexamethasone, ephedrine, fenoterol, flutropium, hydrocortisone, ibudilast, ipratropium, isoprenaline, ketotifen, leukotriene modifiers such as montelukast, zafirlukast and zileuton, mequitazine, nedocromil, orciprenaline, oxitomide, oxitropium, pranlukast hydrate, prednisolone, procaterol, repirinast, salbutamol, seratrodast, sodium cromoglicate, suplatast tosylate, terbutaline, terfenadine, theophylline, tiaramide, tranilast, traxanox, trimetoquinol, tubobuterol, as well as analogs, derivatives, and various salts of the foregoing.

Exemplary agents that produce or stimulate production of nitric oxide include nitrates/nitrites such as nitroglycerin, mioxidil, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds, and L-arginine, as well as analogs, derivatives, and various salts of the foregoing.

Analgesic agents include narcotic and non-narcotic analgesics. Narcotic analgesic agents include alfentanil, allylprodine, alphaprodine, anileridine, benzylmorphine, bezitramide, buprenorphine, butorphanol, clonitazene, codeine, codeine methyl bromide, codeine phosphate, codeine sulfate, desomorphine, dextromoramide, dezocine, diampromide, dihydrocodeine, dihydrocodeinone enol acetate, dihydromorphine, dimenoxadol, dimepheptanol, dimethylthiambutene, dioxaphetyl butyrate, dipipanone, eptazocine, ethoheptazine, ethylmethlythiambutene, ethylmorphine, etonitazene, fentanyl, hydrocodone, hydromorphone, hydroxypethidine, isomethadone, ketobemidone, levorphanol, lofentanil, meperidine, meptazinol, metazocine, methadone hydrochloride, metopon, morphine, myrophine, nalbuphine, narceine, nicomorphine, norlevorphanol, normethadone, normorphine, norpipanone, opium, oxycodone, oxymorphone, papavereturn, pentazocine, phenadoxone, phenazocine, pheoperidine, piminodine, piritramide, proheptazine, promedol, properidine, propiram, propoxyphene, rumifentanil, sufentanil, and tilidine, as well as analogs, derivatives, and various salts of the foregoing.

Non-narcotic analgesics include aceclofenac, acetaminophen, acetaminosalol, acetanilide, acetylsalicylsalicylic acid, alclofenac, alminoprofen, aloxiprin, aluminum bis(acetylsalicylate), aminochlorthenoxazin, 2-amino-4-picoline, aminopropylon, aminopyrine, ammonium salicylate, amtolmetin guacil, antipyrine, antipyrine salicylate, antrafenine, apazone, aspirin, benorylate, benoxaprofen, benzpiperylon, benzydamine, bermoprofen, brofenac, p-bromoacetanilide, 5-bromosalicylic acid acetate, bucetin, bufexamac, bumadizon, butacetin, calcium acetylsalicylate, carbamazepine, carbiphene, carsalam, chloralantipyrine, chlorthenoxazin(e), choline salicylate, cinchophen, ciramadol, clometacin, cropropamide, crotethamide, dexoxadrol, difenamizole, diflunisal, dihydroxyaluminum acetylsalicylate, dipyrocetyl, dipyrone, emorfazone, enfenamic acid, epirizole, etersalate, ethenzamide, ethoxazene, etodolac, felbinac, fenoprofen, floctafenine, flufenamic acid, fluoresone, flupirtine, fluproquazone, flurbiprofen, fosfosal, gentisic acid, glafenine, ibufenac, imidazole salicylate, indomethacin, indoprofen, isofezolac, isoladol, isonixin, ketoprofen, ketorolac, p-lactophenetide, lefetamine, loxoprofen, lysine acetylsalicylate, magnesium acetylsalicylate, methotrimeprazine, metofoline, miroprofen, morazone, morpholine salicylate, naproxen, nefopam, nifenazone, 5′ nitro-2′ propoxyacetanilide, parsalmide, perisoxal, phenacetin, phenazopyridine hydrochloride, phenocoll, phenopyrazone, phenyl acetylsalicylate, phenyl salicylate, phenyramidol, pipebuzone, piperylone, prodilidine, propacetamol, propyphenazone, proxazole, quinine salicylate, ramifenazone, rimazolium metilsulfate, salacetamide, salicin, salicylamide, salicylamide o-acetic acid, salicylsulfuric acid, salsalte, salverine, simetride, sodium salicylate, sulfamipyrine, suprofen, talniflumate, tenoxicam, terofenamate, tetradrine, tinoridine, tolfenamic acid, tolpronine, tramadol, viminol, xenbucin, and zomepirac, as well as analogs, derivatives, and various salts of the foregoing.

Local anesthetic agents include amucaine, amolanone, amylocalne hydrochloride, benoxinate, benzocaine, betoxycaine, biphenamine, bupivacaine, butacaine, butaben, butanilicaine, butethamine, butoxycaine, carticaine, chloroprocaine hydrochloride, cocaethylene, cocaine, cyclomethycaine, dibucaine hydrochloride, dimethisoquin, dimethocaine, diperadon hydrochloride, dyclonine, ecgonidine, ecgonine, ethyl chloride, beta-eucaine, euprocin, fenalcomine, fomocaine, hexylcaine hydrochloride, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine hydrochloride, phenol, piperocaine, piridocaine, polidocanol, pramoxine, prilocalne, procaine, propanocaine, proparacaine, propipocaine, propoxycaine hydrochloride, pseudococaine, pyrrocaine, ropavacaine, salicyl alcohol, tetracaine hydrochloride, tolycaine, trimecaine, and zolamine, as well as analogs, derivatives, and various salts of the foregoing.

Medical devices for use in delivering asthma treatment agents in accordance with the present invention include those that are adapted for implantation or insertion into the trachea or bronchial tree of a patient and from which asthma treatment agents, such as those described above, can be released. Specific examples include tracheal and bronchial stents (including stents having a continuous closed wall structure or a discontinuous open wall structure), grafts, patches, injectable solidifying polymer/drug systems (e.g., intraluminal paving systems), catheters (including, for example, injection catheters, infusion catheters and balloon catheters), guidewires, insertable catheters for aerosol or spray delivery within the airways (e.g., a flexible, insertable spray device analogous to the Blow Mister™ from Estech, Danville, Calif., USA, a device which is presently used for coronary surgery applications) and osmotic pumps.

The asthma treating agents may be delivered to the lungs in a variety of forms. For example, in some aspects of the invention, the therapeutic agent is released from a solid therapeutic-agent-containing region that corresponds to part or the entire medical device. In other aspects of the invention, the therapeutic agent is dispensed from the medical device in association with a fluid.

For example, where the therapeutic-agent is released from a region that corresponds to at least a portion of the medical device (also referred to herein as a “device region”), the device region may correspond, for instance, to the entire medical device, or to one or more portions of the medical device.

For example, the device region(s) can correspond to one or more components of the medical device (e.g., one or more stent struts). As another example, the device regions(s) can be disposed on an underlying medical device substrate, for example, in the form of one or more coating regions, which can cover all or only a portion of the underlying medical device substrate. Substrates include, for example, metallic, ceramic and polymeric substrates. Various metallic, ceramic and polymeric materials appropriate for such substrates are described below. As another example, the device region(s) can correspond to a plurality of particles that are disposed at or near a surface of the medical device, for example, by virtue of their being attached to or embedded in the device surface. Particles used herein are typically microparticles, which may range widely in largest cross-sectional dimension (e.g., the diameter for a spherical particle, the length for a fibrous particle, etc.), for example, ranging from 1 nm to 1500 microns.

Depending on the nature of the therapeutic-agent-containing region(s) employed, the therapeutic agent may be released by any of a number of mechanisms including one or more of the following, among others: diffusion, biodisintegration and compression (e.g., where therapeutic agent is squeezed from a compressible porous region, where therapeutic-agent-containing microcapsules disposed on or within the medical device surface are compressed to the point of rupture, and so forth).

In some embodiments, the device region(s) are porous metallic or ceramic materials from which therapeutic agents are released (e.g., where the medical device is formed from a porous ceramic or metallic material, contains a porous ceramic or metallic component, has a porous ceramic or metallic coating, or comprises porous metallic or ceramic particles). Examples of ceramic materials include silica- and/or calcium-phosphate-based glasses, sometimes referred to as glass ceramics (e.g., silica and bioglass); calcium phosphate ceramics (e.g., hydroxyapatite); metal oxides, including aluminum oxides and transition metal oxides (e.g., oxides of titanium, zirconium, hafnium, tantalum, molybdenum, tungsten, rhenium and iridium); and carbon based ceramic-like materials such as silicon carbides and carbon nitrides. Examples of metals include, for example, noble metals (e.g., silver, gold, platinum, palladium, iridium, osmium, rhodium, titanium, tungsten, and ruthenium) and metal alloys such as cobalt-chromium alloys, nickel-titanium alloys (e.g., nitinol), cobalt-chromium-iron alloys (e.g., elgiloy alloys), nickel-chromium alloys (e.g., inconel alloys), and iron-chromium alloys (e.g., stainless steels, which contain at least 50% iron and at least 11.5% chromium).

In some embodiments, the device region(s) employed for therapeutic agent release are polymeric regions (e.g., where the medical device is formed from a polymeric material, comprises a polymeric component, comprises a polymeric coating, or comprises polymeric particles).

For example, in accordance with some embodiments of the present invention, the polymeric region(s) correspond to polymeric matrices, which are capable of releasing the therapeutic agents via a variety of mechanisms, including diffusion of the therapeutic agents through the matrices and release of the therapeutic agents from the matrices due to disintegration of the matrices. As used herein, a “polymeric matrix” refers to a region that contains at least one polymer and at least one additive, typically, one or more therapeutic agents, although other additives may be present.

Release from a polymeric matrix may be controlled in a number of ways, including the selection of the particular matrix material. For example, numerous polymers appropriate for the polymeric matrices of the present invention are known in the art, including biodisintegrable polymers (i.e., polymers that are dissolved, degraded, resorbed, or otherwise eliminated upon placement in the body) and biostable polymers (e.g., polymers that undergo substantially no biodisintegration during the time that they are intended to reside in the body).

Among biostable polymers are included polyolefins such as polyethylenes (e.g., metallocene catalyzed polyethylenes), polypropylenes, and polybutylenes, polyolefin copolymers, e.g., ethylenic copolymers such as ethylene vinyl acetate (EVA) copolymers, ethylene-methacrylic acid copolymers and ethylene-acrylic acid copolymers, where some of the acid groups can be neutralized with either zinc or sodium ions (commonly known as ionomers); vinyl aromatic polymers such as polystyrene; vinyl aromatic copolymers such as styrene-ethylene-butylene copolymers (e.g., a polystyrene-polyethylene/butylene-polystyrene (SEBS) copolymer, available as Kraton® G series polymers), styrene-isobutylene copolymers (e.g., polystyrene-polyisobutylene-polystyrene (SIBS) copolymers such as those disclosed in U.S. Pat. No. 6,545,097 to Pinchuk) and butadiene-styrene copolymers; polyacetals; chloropolymers such as polyvinyl chloride (PVC); fluoropolymers such as polytetrafluoroethylene (PTFE); polyesters such as polyethyleneterephthalate (PET); polyester-ethers; polyamides such as nylon 6 and nylon 6,6; polyethers; polyamide ethers such as polyether block amides (PEBA); polyoctenamers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Carbothane®, Tecoflex®, Tecothane®, Tecophilic®, Tecoplast®, Pellethane®, Chronothane® and Chronoflex®); silicones; polycarbonates; and blends and additional copolymers (including block, alternating, random and statistical copolymers) of any of the foregoing, among others.

Among biodisintegrable polymers are included polylactic acid, polyglycolic acid and copolymers and mixtures thereof such as poly(L-lactide) (PLLA), poly(D,L-lactide) (PLA); polyglycolic acid [polyglycolide (PGA)], poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), poly(D,L-lactide-co-caprolactone) (PLA/PCL), poly(glycolide-co-caprolactone) (PGA/PCL); polyethylene oxide (PEO), polydioxanone (PDS), polypropylene fumarate, poly(ethyl glutamate-co-glutamic acid), poly(tert-butyloxy-carbonylmethyl glutamate), poly(carbonate-ester)s, polycaprolactone (PCL), polycaprolactone co-butylacrylate, polyhydroxybutyrate (PHBT) and copolymers of polyhydroxybutyrate, poly(phosphazene), poly(phosphate ester), poly(amino acid) and poly(hydroxy butyrate), polydepsipeptides, maleic anhydride copolymers, polyphosphazenes, polyiminocarbonates, poly[(97.5% dimethyl-trimethylene carbonate)-co-(2.5% trimethylene carbonate)], cyanoacrylate, polyethylene oxide, hydroxypropylmethylcellulose, polysaccharides such as hyaluronic acid, chitosan and regenerate cellulose, and proteins such as gelatin and collagen, as well as blends and additional copolymers of the foregoing, among others.

Release from a matrix may also be controlled, for example, by varying the thickness of the matrix or by varying the porosity of the matrix (e.g., an additional component may be added to a matrix system to increase its porosity).

Another way of controlling release is to utilize multiple layers of polymeric materials. For example, transport from a matrix material containing a therapeutic agent may be reduced by providing another material (which may or may not contain therapeutic agent) in the form of a barrier layer over the matrix material. Barrier layers may also be used in cases where it is desirable to effectively block diffusion from a less desirable surface of a matrix, directing diffusion to other more desirable surfaces. For example, a barrier layer may be provided on the inside surface of a matrix in the form of a stent, directing diffusion to the outer surface.

In some embodiments, multiple matrices with differing release characteristics are provided, for example, to provide near-, intermediate- and long-term release characteristics, respectively. These matrices may be, for example, stacked on top of one another, or placed laterally relative to one another.

In some embodiments of the invention, the therapeutic agent is provided within polymeric microparticles, for example, provided within a polymeric matrix (also referred to herein as micromatrices) or encapsulated by a polymeric shell (also referred to herein as microcapsules).

For example, the therapeutic agent may be released from the microparticles in these embodiments by diffusion and/or degradation, in which case microparticles populations with differing release characteristics may be provided, for example, to provide near-, intermediate- and long-term release characteristics.

As another example, the therapeutic agent may be released by bursting the microparticles. For example, therapeutic agent filled microcapsules may be provided on or within a medical device such that they are crushed between components or the medical device (e.g., between an inner inflation balloon and an outer porous balloon) or between a component of the device and surrounding tissue (e.g., between a balloon and a surrounding lung tissue), thereby releasing the therapeutic agent within the microcapsules.

Those of ordinary skill in the art can readily form therapeutic-agent-containing ceramic, metallic and polymeric device regions such as those described above using methods that are well known in the art.

In other aspects of the invention, therapeutic agents are delivered from the medical devices of the present invention in association with a fluid.

The therapeutic agent can be, for example, dissolved in the fluid, or provided within the fluid in association with a dispersed phase, for instance in association with (a) microparticles (including, particles of the therapeutic agent, micromatrices containing the therapeutic agent, and microcapsules containing the therapeutic agent) or (b) liposomes or emulsions (e.g., where the therapeutic agent is a substantially water-insoluble liquid or wherein it is dissolved in a substantially water-insoluble liquid).

The therapeutic-agent-containing fluids may include various adjuvants, including water and/or organic solvents, which may optionally contain additional adjuvants as desired, for example, salts and/or buffers to establish a desired tonicity and/or pH, viscosity adjusting agents, crosslinkable polymers and crosslinking agents, surface active agents (including phospholipids, block copolymers, and other biologically compatible surfactants), and so forth. In some embodiments, the fluid that is delivered is a viscous fluid or a gel (gels typically act as viscous fluids once an applied stress exceeds a critical value, frequently referred to as the yield stress, which causes them to flow).

Those of ordinary skill in the art can readily form therapeutic-agent-containing fluids such as those described above using methods that are well known in the art.

In still other aspects of the invention, therapeutic agents are delivered, in association with fluids, from medical devices (e.g., catheters), after which the administered fluid solidifies at the site of administration. For example, as described in U.S. Patent Application Publication No. 20020037358 to Barry et al., therapeutic agent may be administered in association with an intraluminal paving system. As described therein, a polymeric materialitherapeutic agent matrix is typically applied directly to an interior surface of a lumen (in the present case, the trachea or a branch of the bronchial tree). The intraluminal paving system may be formed, for example, by admixing a therapeutic agent with a fluid polymer composition, typically in the absence of a solvent, to form a fluid polymer/therapeutic agent mixture. This mixture is then applied directly to the luminal surface by any known application method, for example, by injecting the mixture against the luminal surface. Solidification of the mixture occurs in-situ. To facilitate solidification, a cross-linking or curing agent may be added to the mixture prior to application thereof to the luminal surface. Solidification may also be facilitated in-situ (a) upon contact with bodily fluids such as water that are present at the site where the mixture is applied to the luminal surface, or (b) by exposing the polymer/therapeutic agent mixture, after application to the luminal surface, to curing radiation such as ultraviolet radiation, laser light, or heat. The polymeric material incorporated into the paving system may be either biodisintegrable or biostable.

The ability to non-invasively image regions where the therapeutic agents are introduced (or where they have previously been introduced) is a valuable diagnostic tool for the practice of the present invention. Among such currently available non-invasive imaging techniques are included magnetic resonance imaging (MRI), ultrasonic imaging, x-ray fluoroscopy, nuclear medicine, and others. To assist with non-invasive imaging, in some embodiments, the medical devices described herein are provided with one or more regions which have enhanced contrast (e.g., the medical devices can be provided with marker regions of enhanced contrast or the entire medical device can be provided with enhanced contrast). In some embodiments, the therapeutic-agent-containing compositions described herein are provided with enhanced contrast. For example, various imaging contrast agents (i.e., substances that enhance the image produced by medical diagnostic equipment) are known, including x-ray contrast agents (such as iodinated compounds, metals, metal salts and metal oxides, e.g., bismuth salts and oxides), ultrasonic contrast agents (such as echogenic and echolucent particles, including micro-bubbles and inorganic and organic particles, e.g., calcium carbonate, hydroxyapatite, silica, polylactic acid, and polyglycolic acid), and MRI contrast agents (e.g., gadolinium(III) and materials containing the same, such as gadolinium(III)-containing chelates).

As noted above, in various aspects of the invention, a medical device is implanted or inserted into the patient, after which therapeutic agent is released from the medical device into the patient over a period of time. In these aspects, release profiles may vary widely and may be selected, for example, from combinations of the following: a cumulative therapeutic agent release selected from at least 5%, at least 10%, at least 15%, at least 20%, at least 25%, at least 30%, at least 40%, at least 50%, at least 60%, at least 70%, at least 80%, at least 90%, at least 95%, and at least 99% (relative to the total therapeutic agent in the medical device), after implantation or insertion of the device for a period selected from 1 day, 2 days, 4 days, 1 week, 2 weeks, 1 month, 2 months, 4 months, 1 year and 2 years.

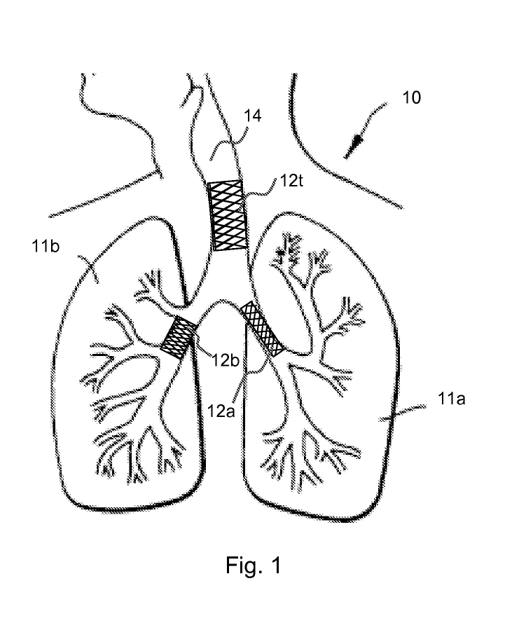

One particular embodiment of the invention will now be described in conjunction with FIG. 1, which schematically illustrates the trachea 14, left lung 11a and right lung 11b of a patient 10. Although myriad placement positions are possible, the embodiment of FIG. 1 schematically illustrates: (a) a first stent 12t, positioned in the trachea 12t, (b) a second stent 12a, positioned in the primary bronchus of the left lung 11a, and (c) a second stent 12b, positioned in the primary bronchus of the right lung 11b. In addition, although myriad stent designs are possible, in the embodiment of FIG. 1, the particular stents shown therein are beneficially formed of a metallic material, which is coated with a polymeric matrix from which the therapeutic agent is released.

In various other aspects of the invention, the therapeutic agent is released from a medical device in association with a liquid. For example, in some embodiments, liquid compositions are administered to patients by directing the liquid onto the lumen surface (e.g., the inner surface of the trachea or bronchial tree), for instance, by spraying, extruding or otherwise dispensing the liquid from the medical device. As a specific example, an infusion balloon may be used in which fluid between an inner inflatable balloon and an outer porous balloon is forced through the porous outer balloon upon inflation of the inner balloon. As another specific example, a device such as a catheter can be inserted into the bronchial tree for aerosol or spray delivery of fluid within the lungs. As yet another specific example, microcapsules containing therapeutic agent disposed at or near the medical device surface (e.g., on an inflation balloon surface, or between an inner inflation balloon and a porous outer balloon) may be compressed to a point where the microcapsules rupture and release the therapeutic agent therein. Where a balloon is used to administer the therapeutic agent, the balloon may be further provided with cutting blades as is known in the art to relieve constriction. In some embodiments, therapeutic agent-containing liquid may be injected into the lumen tissue (e.g., using an injection catheter).

Although various embodiments are specifically illustrated and described herein, it will be appreciated that modifications and variations of the present invention are covered by the above teachings and are within the purview of the appended claims without departing from the spirit and intended scope of the invention.

What is claimed is:

1. A method for the treatment of asthma, comprising:
providing an implantable or insertable medical device that comprises an asthma treatment agent; and
inserting or implanting the medical device within the trachea or the bronchial tree of a patient, whereupon the therapeutic agent is delivered to the patient in an amount effective to reduce or eliminate the symptoms of asthma.

2. The method of claim 1, wherein said therapeutic agent is selected from steroidal and non-steroidal anti-inflammatory agents a glucocorticoid, an antiproliferative agent, a muscle relaxant, an anti-macrophage agent, nitric oxide donors and agents that stimulate production of nitric oxide in vivo.

3-7. (canceled)

8. The method of claim 1, wherein said therapeutic agent is delivered from a polymeric region, metallic region or ceramic region of said medical device.

9. The method of claim 8, wherein said polymeric region is a biostable polymeric region or a biodisintegrable polymeric region.

10. (canceled)

11. The method of claim 8, wherein said polymeric region is a polymeric matrix disposed as a coating on a medical device substrate or is a stand-alone polymeric matrix.

12. The method of claim 11, wherein said medical device is selected from a stent, a patch, and a graft.

13-15. (canceled)

16. The method of claim 8, wherein said polymeric region of said medical device is a polymeric microparticle disposed at or near the surface of said medical device.

17-18. (canceled)

19. The method of claim 1, wherein a fluid comprising said therapeutic agent is forced from said medical device.

20. The method of claim 19, wherein said medical device is a catheter.

21. The method of claim 20, wherein said catheter comprises a balloon, and wherein said therapeutic agent is released upon expansion of said balloon.

22. The method of claim 21, wherein said catheter comprises an infusion balloon, wherein said catheter comprises a porous compressible coating that further comprises said therapeutic agent, wherein said catheter comprises microcapsules which are adapted to be ruptured upon expansion of said balloon, or wherein said balloon comprises cutting blades.

23-25. (canceled)

26. The method of claim 19, wherein said fluid comprising said therapeutic agent solidifies subsequent to contact with the trachea or the bronchial tree of said patient.

27. The method of claim 1, wherein said medical device is selected from a patch, a graft, a stent and a paving system.

28-29. (canceled)

30. The method of claim 1, wherein the cumulative release of said therapeutic agent is in an amount selected from at least 5%, least 10%, least 15%, least 20%, least 25%, least 30%, least 40%, least 50%, least 60%, least 70%, least 80%, least 90%, least 95%, and least 99%, relative to the total therapeutic agent in the medical device, after implantation or insertion at said site for a period selected from 1 day, 2 days, 4 days, 1 week, 2 weeks, 1 month 2 months, 4 months, 1 year and 2 years.

31. (canceled)

32. The method of claim 20, wherein said catheter is configured for aerosol or spray delivery of said fluid.

33. A medical device comprising an asthma treatment agent selected from steroidal anti-inflammatory agents, non-steroidal anti-inflammatory agents, antiproliferative agents, muscle relaxants, anti-macrophage agents, nitric oxide donors, agents that stimulate production of nitric oxide in vivo, cGMP activators and anti-histamines, said medical device being configured for implantation or insertion within the trachea or the bronchial tree of a patient, whereupon the asthma treatment agent is delivered to the patient in an amount effective to reduce or eliminate the symptoms of asthma.

34. (canceled)

35. The medical device of claim 33, wherein said asthma treatment agent is delivered from a metallic region, ceramic region, or polymeric region of said medical device.

36. The medical device of claim 35, wherein said polymeric region is a biostable polymeric region or a biodisintegrable polymeric region.

37. (canceled)

38. The medical device of claim 35, wherein said polymeric region is a stand-alone polymeric matrix or is a polymeric matrix disposed as a coating on a medical device substrate.

39. (canceled)

40. The medical device of claim 35, wherein said polymeric region is a polymeric microparticle disposed at or near the surface of said medical device.

41-42. (canceled)

43. The medical device of claim 33, wherein said medical device is selected from a stent, a patch, a graft, a balloon catheter, and a paving system.

44. The medical device of claim 43, wherein said balloon comprises a porous compressible coating that further comprises said therapeutic agent, wherein said balloon comprises microcapsules which are adapted to be ruptured upon expansion of said balloon, or wherein said balloon comprises cutting blades.

45-47. (canceled)

48. A kit comprising (a) a composition comprising an asthma treatment agent selected from steroidal anti-inflammatory agents, non-steroidal anti-inflammatory agents, antiproliferative agents, muscle relaxants, anti-macrophage agents, nitric oxide donors, agents that stimulate production of nitric oxide in vivo, cGMP activators and anti-histamines, and (b) a medical device configured for implantation or insertion within the trachea or the bronchial tree of a patient and for delivery of said composition to a patient in an amount effective to reduce or eliminate the symptoms of asthma.

49. The kit of claim 48, wherein said composition is a fluid comprising said asthma treatment agent and wherein said medical device is a catheter.
